(19) United States
(12) Patent Application Publication
(10) Pub. No.: US 2021/0128243 A1
(43) Pub. Date: May 6, 2021

(54) AUGMENTED REALITY METHOD FOR ENDOSCOPE

(71) Applicant: CHANG BING SHOW CHWAN MEMORIAL HOSPITAL, Lukang Township (TW)

(72) Inventors: Atul KUMAR, Lukang Township (TW); Yen-Yu WANG, Lukang Township (TW); Sheng-Lei YAN, Lukang Township (TW); Kai-Che LIU, Lukang Township (TW); Shih-Wei HUANG, Lukang Township (TW); I-Chun LEE, Lukang Township (TW); Wan-Chi HUNG, Lukang Township (TW)

(21) Appl. No.: 16/672,010

(22) Filed: Nov. 1, 2019

(51) Int. Cl. A61B 34/10; A61B 90/00; G06T 19/00; G06F 3/01

(52) U.S. Cl. CPC A61B 34/10; A61B 90/36; A61B 2034/2065; G06F 3/011; A61B 2090/365; G06T 19/006

(57) ABSTRACT

An augmented reality method for an endoscope includes constructing a first virtual three-dimensional model by using the volume image; setting a reference frame of a position tracking device as a global reference frame; obtaining a second virtual three-dimensional model of the subject by using laser scanning; calculating a first transformation between the first virtual three-dimensional model and the second virtual three-dimensional model by the iterative closest point algorithm, and applying the first transformation to the first virtual three-dimensional model to generate a third virtual three-dimensional model; tracking the first tracker by the position tracking device to provide an endoscopic virtual position; imaging a virtual image corresponding to an endoscopic image imaged by the endoscope based on the endoscopic virtual position and the third virtual three-dimensional model, and superimposing the endoscopic image with the virtual image to display a superimposed image.

### BACKGROUND OF THE INVENTION

### 1. Field of the Invention

The present invention relates to an augmented reality method, more particularly to an augmented reality method and system for an endoscope.

### 2. Description of the Related Art

Conventionally, surgical anatomy is typically visualized as a 2D image on a screen produced with the help of a camera and an optical system passed through the small incisions or natural orifices on a patient's body during endoscopic surgery. Special surgical equipment is further introduced into the body through small incisions to perform the operation. Ideally, endoscopic surgery may cause less tissue injury compared to open surgery. This therefore helps patients in rapid recuperation with less pain after surgery. However, when operating endoscopic surgery, a surgeon may only perform anatomy with a narrow visual field. Moreover, the 2D image of the conventional endoscope may not provide depth perception of the visual field. An inadvertent injury may easily occur during surgery if the surgeon is not well experienced.

Augmented reality (AR) is a technology that superimposes a computer-generated image on a user's visual field of the real world, thus providing a composite view. Various methods of applying AR to the visualization of endoscope have been carried out to enhance anatomical structures displayed on the video of the endoscope. However, these methods are still immature in the aspects of model building, alignment, and tracking in terms of anatomy.

Hence, there is still a need for a method capable of combining the 3D message of a virtual model with an endoscopic image to help the surgeon easily view the structure of the posterior surface of the organ.

### SUMMARY OF THE INVENTION

The present invention aims to provide an augmented reality method and system that may combine the image of a virtual 3D model of the patient with the endoscopic image in real-time along with a real-time display of the relevant instruments for endoscopic surgery.

One aspect of the present invention provides an augmented reality method for an endoscope, including: obtaining a volume image of a subject and constructing a first virtual three-dimensional model by using the volume image; setting a reference frame of a position tracking device as a global reference frame; obtaining a second virtual three-dimensional model of the subject by using laser scanning and registering the second virtual three-dimensional model to the global reference frame; aligning the first virtual three-dimensional model with the global reference frame, matching the first virtual three-dimensional model with the second virtual three-dimensional model by an iterative closest point algorithm (ICP) in order to calculate a first transformation, and applying the first transformation to the first virtual three-dimensional model to generate a third virtual three-dimensional model on a render window; constructing an endoscopic virtual model based on geometrical parameters of an endoscope mounted with a first tracker, and tracking the first tracker by the position tracking device to provide an endoscopic virtual position; and moving the endoscopic virtual model on the render window to the endoscopic virtual position, imaging a virtual image corresponding to an endoscopic image imaged by the endoscope based on the endoscopic virtual position and the third virtual three-dimensional model, and superimposing the endoscopic image imaged by the endoscope with a virtual image to display a superimposed image.

Preferably, the volume image is an image imaged by means of CT or MRI.

Preferably, a specific area in the volume image is performed with segmentation and images of the specific area which is segmented are stacked to form the first virtual three-dimensional model, and the first virtual three-dimensional model is registered to the global reference frame.

Preferably, the relatively static surface of the subject is obtained by laser scanning to construct a second virtual three-dimensional model, and the second virtual three-dimensional model is registered to the global reference frame.

Preferably, before the first virtual three-dimensional model is matched with the second virtual three-dimensional model, a local reference frame is established at a center of the first virtual three-dimensional model, and the local reference frame is aligned with the global reference frame.

Preferably, the method further includes displaying a relative position of the endoscopic virtual model to the third virtual three-dimensional model on the render window based on the endoscopic virtual position and superimposing the endoscopic image with the virtual image to display a superimposed image.

Preferably, the method further includes constructing a surgical instrument virtual model based on geometrical parameters of a surgical instrument mounted with a second tracker, tracking the second tracker by the position tracking device in order to provide a surgical instrument virtual position, and displaying a relative position of the surgical instrument virtual model to the third virtual three-dimensional model on the render window based on the surgical instrument virtual position.

Preferably, the method further includes photographing an endoscopic calibration tool having a plurality of marked points by using the endoscope to image the plurality of marked points, identifying the plurality of marked points by a computer algorithm to calculate an intrinsic parameter of the endoscope, and adjusting parameters of a virtual camera on the render window by using the intrinsic parameter.

Preferably, the endoscopic calibration tool is a hemisphere tool, and the plurality of marked points is marked on a curved surface of the hemisphere tool.

When the method of the present invention is applied to the augmented reality of endoscopic surgery, the 3D message from the virtual model of the organ may be used to enhance the endoscopic image. In the view of the endoscopic augmented reality, the surgeon may see the structure of the posterior surface of the organ. This helps the less experienced surgeon avoid the damage to the structure of the posterior surface. The virtual model further provides messages of adjacent structures that are usually outside the visual field of the endoscope to improve the operability when the surgeon operates the augmented reality.

### BRIEF DESCRIPTION OF THE DRAWINGS

FIG. 1 depicts a flow chart of the augmented reality method for an endoscope according to the present invention.

FIG. 2 depicts a block diagram of the augmented reality system for an endoscope according to the present invention.

FIG. 3 depicts a schematic diagram of constructing the three-dimensional model of a preoperative organ in the augmented reality method for an endoscope according to the present invention.

FIG. 4 depicts a schematic diagram of aligning the position tracking device in the augmented reality method for an endoscope according to the present invention.

FIG. 5 depicts a schematic diagram of the iterative closest point algorithm of the augmented reality method for an endoscope according to the present invention.

FIG. 6 depicts a schematic diagram of the endoscope mounted with the first tracker according to the present invention.

FIG. 7 depicts a schematic diagram of the surgical instrument mounted with the second tracker according to the present invention.

FIG. 8 depicts a schematic diagram of constructing the real-time virtual three-dimensional model in the augmented reality method for an endoscope according to the present invention.

FIG. 9 depicts a schematic diagram for aligning the local reference frame of the three-dimensional model of a preoperative organ and the global reference frame in the augmented reality method for an endoscope according to the present invention.

FIG. 10 depicts a schematic diagram for aligning the three-dimensional model of a preoperative organ and the real-time virtual three-dimensional model in the augmented reality method for an endoscope according to the present invention.

FIG. 11 depicts a schematic diagram of using an endoscopic calibration tool having different marked points to calibrate an endoscope.

FIG. 12 depicts an image of the superimposition of the endoscopic image and the virtual three-dimensional model according to the present invention.

### DESCRIPTION OF THE PREFERRED EMBODIMENTS

To make the aforementioned purpose, the technical features, and the gains after actual implementation more obvious and understandable to a person of ordinary skill in the art, the following description shall be explained in more detail with reference to the preferable embodiments together with related drawings.

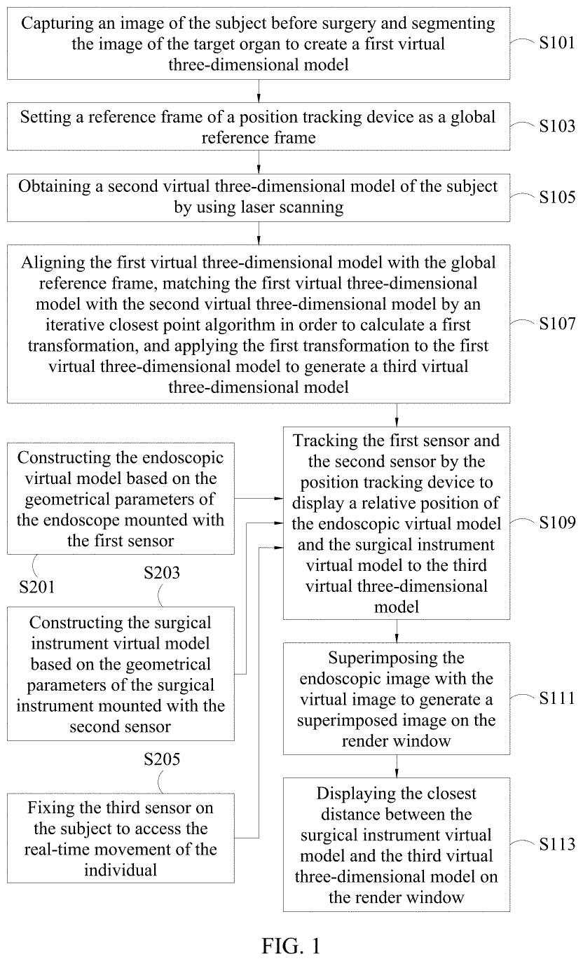

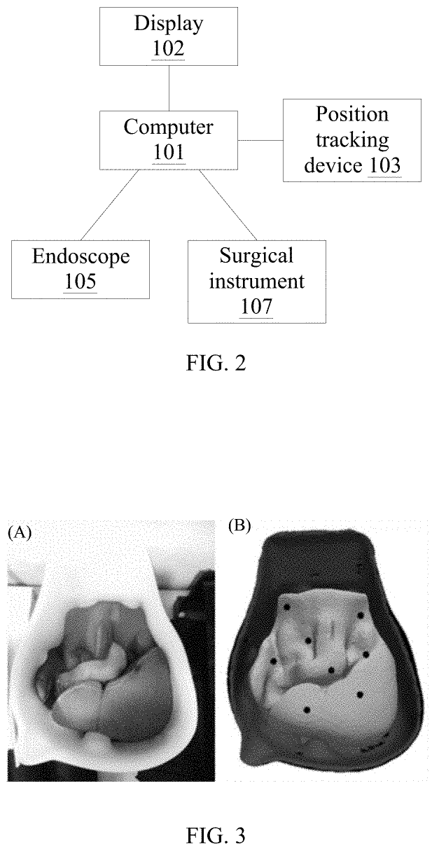

Please refer to FIG. 1 and FIG. 2 which respectively depict a flow chart of the augmented reality method and a block diagram of the system for an endoscope according to the present invention.

In step S101, preoperative volume imaging of the subject is acquired. The subject may be a human. The preoperative volume image may be from tomographic imaging, magnetic resonance imaging, or any preoperative volume imaging technique known to a person of ordinary skill in the art. The preoperative volume image of the subject obtained by the aforementioned manner is input to the computer 101 to construct the first virtual three-dimensional model which is displayed on the render window of the display 102. The first virtual three-dimensional model may be used as a three-dimensional model of the preoperative organ.

In step S103, the global reference frame is created by the position tracking device 103, and the global reference frame is then registered in the computer 101.

In step S105, the relatively static surface of the subject is obtained by laser scanning to construct a second virtual three-dimensional model, and the second virtual three-dimensional model is registered to the global reference frame and displayed on the render window of the display 102. In an embodiment, the second virtual three-dimensional model may be used as a real-time virtual three-dimensional model.

In step S107, the first virtual three-dimensional model is aligned with the global reference frame. The first virtual three-dimensional model is matched with the second virtual three-dimensional model by the iterative closest point algorithm to make the two models position in the same frame so as to calculate the first transformation between the two models. The computer 101 applies the first transformation to the first virtual three-dimensional model to generate a third virtual three-dimensional model on the render window.

In step S201, the endoscopic virtual model is constructed based on the geometrical parameters of the endoscope 105 mounted with the first sensor. Specifically, a virtual model including the endoscope 105 and the first sensor is constructed as a virtual model of the endoscope by the known geometrical parameters of the endoscope 105 and the first sensor, such as length, width, height, and other specific size parameters. The endoscopic virtual model is displayed on the render window of display 102.

In step S203, the surgical instrument virtual model is constructed based on the geometrical parameters of the surgical instrument 107 mounted with the second sensor. Specifically, a virtual model including the surgical instrument 107 and the second sensor is constructed as the surgical instrument virtual model by the known geometrical parameters of the surgical instrument 107 and the second sensor, such as length, width, height, and other specific size parameters. The surgical instrument virtual model is then displayed on the render window of display 102.

In step S205, before the surgery, the third sensor is fixed on the subject to access the real-time movement of the subject.

In step S109, the first sensor mounted on the endoscope 105 and the second sensor mounted on the surgical instrument 107 are tracked by the position tracking device 103 to respectively obtain the endoscopic virtual position and the surgical instrument virtual position. Specifically, since the global reference frame is created based on the position tracking device 103, the endoscopic virtual model and the surgical instrument virtual model are registered to the global reference frame based on the endoscopic virtual position and the surgical instrument virtual position. Thus, the relative position of the endoscopic virtual model and the surgical instrument virtual model to the third virtual three-dimensional model may be displayed on the render window.

In step S111, the endoscopic image imaged by the endoscope 105 is superimposed with the virtual image corresponding to the endoscopic image on the render window to generate a superimposed image, wherein the virtual image is imaged based on the third virtual three-dimensional model. This enables the surgeon to view both the endoscopic image and the virtual three-dimensional model in the render window.

In step S113, the computer 101 calculates the closest distance between the surgical instrument virtual model and the third virtual three-dimensional model, and the closest distance is shown in the superimposed image of the render window such that the surgeon determines a relative position of the surgical instrument to the organ in real-time.

In short, the augmented reality method for an endoscope of the present invention may achieve the purpose of combining endoscopic images with virtual three-dimensional models of organs by introducing preoperative volume images and real-time images into the same frame and constructing an integrated virtual three-dimensional model. Further, the present invention provides a relative position of the surgical instrument to the virtual three-dimensional model of the organ so that the surgeon may obtain the structure of the posterior surface of the organ to avoid damage to the structure of the posterior surface.

Hereafter, the augmented reality method for an endoscope of the present invention is further described by means of specific examples.

Please refer to FIG. 2. The augmented reality system for an endoscope includes a computer 101, a display 102, a position tracking device 103, an endoscope 105, a surgical instrument 107, a scanning device for scanning the preoperative volume of the subject, and a laser scanner. A virtual three-dimensional model may be constructed by the system of the present invention and a virtual view may be displayed on the display.

Please refer to FIG. 3 which depicts a schematic diagram of constructing the first virtual three-dimensional model in the augmented reality method for an endoscope according to the present invention. The CT scanning and volume imaging image of MRI to a subject before the surgery are segmented into different organs (including skin and bone). The segmented image is stacked using the volume reconstruction algorithm to reconstruct a three-dimensional model of the preoperative organ (referring to Lorensen, William E. and Harvey E. Cline “Marching cubes: A high resolution 3D surface construction algorithm” ACM siggraph computer graphics. Vol. 21, No. 4. ACM, 1987). All organs are preserved as separate files with their own names. In the embodiment of the present invention, (A) of FIG. 3 is a model of the subject; (B) is a three-dimensional model of a preoperative organ segmented and reconstructed using images obtained by CT scanning.

The global reference frame: The reference frame of the position tracking device 103 is considered the global reference frame of the system.

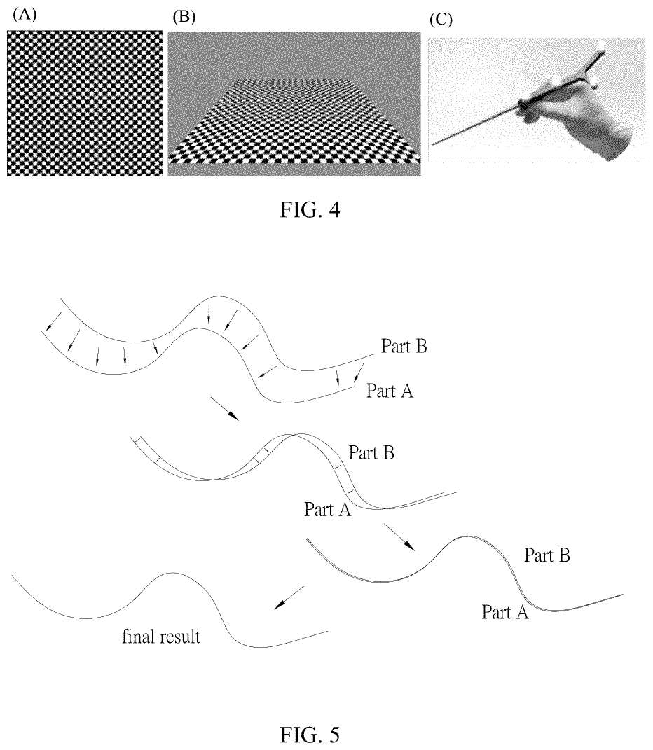

The alignment of the reference frame of the laser scanner: Please refer to FIG. 4 and FIG. 5 which depict a schematic diagram of aligning the position tracking device in the augmented reality method for an endoscope according to the present invention and a schematic diagram of the iterative closest point algorithm of the augmented reality method for an endoscope according to the present invention. Wherein, (A) of FIG. 4 shows a checkerboard fixed on a plane, (B) shows a 3D checker model of the position tracking device, and (C) shows a 3D positioning recorder mounted with a tracker. Firstly, three or more checker points (ChekerPoints_{G}) of the checkerboard in the reference frame of the position tracking device are recorded by using the 3D positioning recorder of the position tracking device. Next, the checkerboard is scanned with a laser scanner to create a 3D checker model (CheckerModel_{L}) having a checkerboard structure. A mouse is used to click on the 3D checker model displayed in the window to identify the corner points corresponding to the checker points in the 3D checker model. Corresponding point pairs are employed to calculate the transformation (T_{L2G}) of the laser scanner of the position tracking device by using the iterative closest point algorithm (ICP).

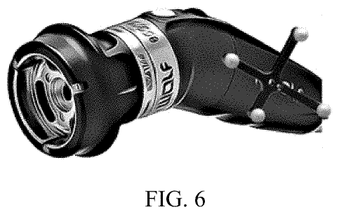

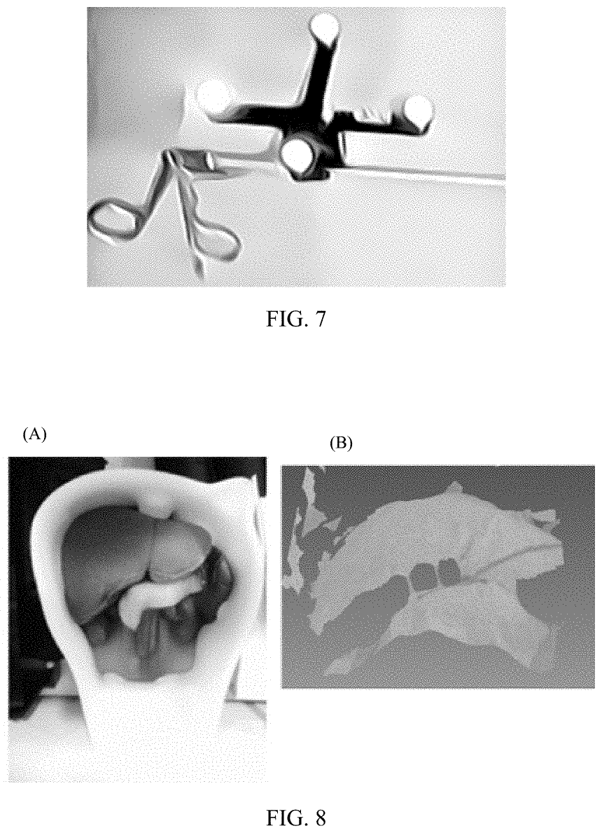

The endoscope and the surgical instrument virtual model: Please refer to FIG. 6 and FIG. 7 which respectively depict schematic diagrams of the endoscope mounted with the first tracker and the surgical instrument mounted with the second tracker according to the present invention. As shown in FIG. 6 and FIG. 7, the first tracker and the second tracker are respectively fixed to the endoscope camera head and the surgical instrument. Since the geometrical parameters of the surgical instrument, the endoscope camera head, and the tracker are known, the endoscopic virtual model and the surgical instrument virtual model may be separately constructed and displayed on the render window of the display based on the geometric structure of the surgical instrument and the endoscope. Further, the local reference frame of the first tracker and the second tracker is known from the manual of the manufacturer, and the positions of the surgical instrument and the endoscope are also known by the position tracking device tracking the positioning of the first tracker and the second tracker. The first tracker and the second tracker may be moved in the render window by the rigid body transformation recorded by the position tracking device such that the endoscope connected to the first tracker and the surgical instrument connected to the second tracker may also be moved by the same transformation.

Constructing and scanning a real-time virtual three-dimensional model with a laser scanner: Please refer to FIG. 8 which depicts a schematic diagram of constructing the real-time three-dimensional model in the augmented reality method for an endoscope according to the present invention, wherein (A) of FIG. 8 is a model of the subject; (B) is a real-time three-dimensional model constructed and scanned by using the laser scanner. Before the surgery, the laser scanner is used to create a real-time virtual three-dimensional model of the relatively static surface (for instance, having bony landmarks such as sternum and clavicle) of the subject.

Registration of the preoperative organ three-dimensional model and the real-time virtual three-dimensional model:

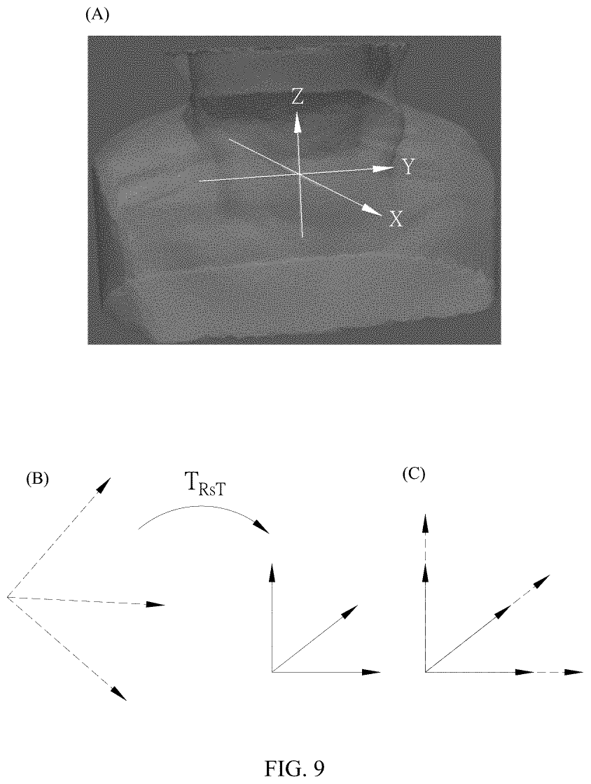

A. Initial alignment: Please refer to FIG. 9 which depicts a schematic diagram for aligning the local reference frame of the three-dimensional model of a preoperative organ and the global reference frame in the augmented reality method for an endoscope according to the present invention. Firstly, please refer to FIG. 9 (A), the first surface model (Model_{CT1}) is set in the three-dimensional model of the preoperative organ, and a local reference frame is created at the center of the first surface model (Model_{CT1}) such that the original point is in the center of the first surface model; where z-axis is toward the head of the subject, x-axis toward the ceiling, and y-axis toward the inner side of the subject. Next, please refer to FIG. 9 (B). The transformation between the local reference frame (dotted-line reference frame) of the first surface model (Model_{CT1}) and the global reference frame (solid-line reference frame) is calculated according to TR_{sT}. Please refer to FIG. 9 (C). The first surface model (Model_{CT1}) is transformed into the second surface model (Model₀₋₂) aligned with the global reference frame by using TR_{sT}.

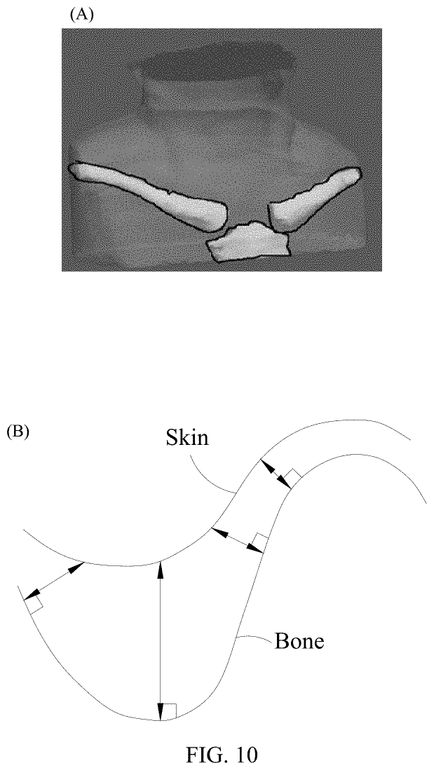

B. Registration refinement: Please refer to FIG. 10 which depicts a schematic diagram for aligning the three-dimensional model of a preoperative organ and the real-time virtual three-dimensional model in the augmented reality method for an endoscope according to the present invention. Firstly, the real-time virtual three-dimensional model scanned by the laser scanner is input in the software. As shown in FIG. 10 (A), the software finds out the closest bone of the real-time virtual three-dimensional model in the second surface model (Model_{CT2}) and calculates the vertical distance between the bone and the outer skin surface. Next, as shown in FIG. 10 (B), the smallest values of the vertical distance from the skin in the second surface model (Model_{CT2}) is selected. The surface of the area in the second surface model is aligned with the surface of the real-time virtual three-dimensional model constructed by the laser scanner, and the iterative closest point algorithm is used to align two surfaces and calculate their transformation. Then, the transformation is applied to the second surface model (Model_{CT2}) to construct the third virtual three-dimensional model that is eventually used.

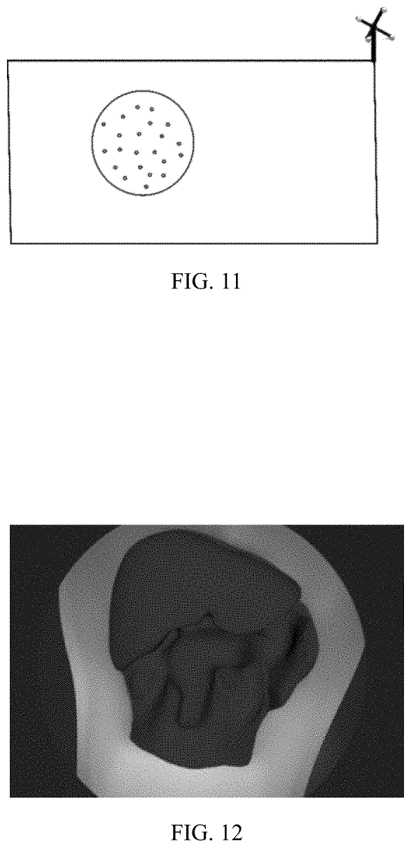

Registration of the endoscope camera head: Please refer to FIG. 11 which depicts a schematic diagram of using an endoscopic calibration tool having different marked points to calibrate an endoscope. As shown in FIG. 11, the camera calibration tool and endoscope camera positioning in the global reference frame are realized by a hemisphere tool made of solid material having more than 8 different marked points (Landmarksₛₚₕₑᵣₑ), the marked points are on the curved surface of the hemisphere tool, and the flat surface of the hemisphere tool is attached to a tray. A tracker is also attached to one of the corners of the tray. The 3D position (Landmarks3Dₛₚₕₑᵣₑ) of the center of the marked points is recorded using a position tracking probe. An image of the tray is captured with the endoscope camera mounted with the first tracker so that at least 8 marked points on the hemisphere tool are shown. At least 8 marked points (Landmarksₛₚₕₑᵣₑ) are identified in the image with the computer algorithm (Ref: Moon, Hankyu, Rama Chellappa, and Azriel Rosenfeld. “Optimal edge-based shape detection.” IEEE Transactions on Image Processing 11.11 (2002): 1209-1227.), and their respective 2D pixel co-ordinates (Landmarks2Dₛₚₕₑᵣₑ) are recorded. The intrinsic parameters and extrinsic parameters of the endoscope camera are calculated with a computer program (Triggs, Bill. “Camera pose and calibration from 4 or 5 known 3d points.” 7th International Conference on Computer Vision (ICCV'99). Vol. 1. IEEE Computer Society, 1999.) using the 3D position (Landmarks3Dₛₚₕₑᵣₑ) and the 2D pixel coordinates (Landmarks2Dₛₚₕₑᵣₑ). The calculated intrinsic parameter is used to adjust the virtual camera parameters in the render window. The position of the first tracker (Cameraₜₒₒₗ) mounted on the endoscope camera and the extrinsic parameters (Camera_{Extrinsic}) are also recorded. A transformation (CamTool2Sensor_{T}) between the first tracker (Camera_{Tool}) on the endoscope camera and the extrinsic parameter (Camera_{Extrinsic}) is calculated.

Tracking and displaying: Please refer to FIG. 12 which depicts an image of the superimposition of the endoscopic image and the virtual three-dimensional model according to the present invention. As the system operates, the first tracker on the endoscope camera head provides the position which is further transformed by CamTool2Sensor_{T} to obtain the endoscopic virtual position. The virtual camera in the render window is moved to the endoscopic virtual position and a virtual image (ImageV) is imaged by a virtual camera. Thus, as shown in FIG. 12, the endoscopic image captured by the endoscope camera (ImageR) is superimposed with the virtual image (ImageV) to display a superimposed image. The surgical instrument virtual model is moved based on the position of the second tracker recorded by the position tracking device.

The present invention has specifically described the augmented reality method and system for an endoscope in the aforementioned embodiment. However, it is to be understood by a person of ordinary skill in the art that modifications and variations of the embodiment may be made without departing from the spirit and scope of the present invention. Therefore, the scope of the present invention shall be described as in the following claims.

What is claimed is:

1. An augmented reality method for an endoscope, comprising:
obtaining a volume image of a subject and constructing a first virtual three-dimensional model by using the volume image;
setting a reference frame of a position tracking device as a global reference frame;
obtaining a second virtual three-dimensional model of the subject by using laser scanning and registering the second virtual three-dimensional model to the global reference frame;
aligning the first virtual three-dimensional model with the global reference frame, matching the first virtual three-dimensional model with the second virtual three-dimensional model by an iterative closest point algorithm in order to calculate a first transformation, and applying the first transformation to the first virtual three-dimensional model to generate a third virtual three-dimensional model on a render window;
constructing an endoscopic virtual model based on geometrical parameters of the endoscope mounted with a first tracker, and tracking the first tracker by the position tracking device to provide an endoscopic virtual position; and
moving the endoscopic virtual model on the render window to the endoscopic virtual position, imaging a virtual image corresponding to an endoscopic image imaged by the endoscope based on the endoscopic virtual position and the third virtual three-dimensional model, and superimposing the endoscopic image imaged by the endoscope with the virtual image to display a superimposed image.

2. The method according to claim 1, wherein the volume image is an image imaged by means of CT or Mill.

3. The method according to claim 2, wherein a specific area in the volume image is performed with segmentation and images of the specific area which is segmented are stacked to form the first virtual three-dimensional model, and the first virtual three-dimensional model is registered to the global reference frame.

4. The method according to claim 1, a relatively static surface of the subject is obtained by laser scanning to construct the second virtual three-dimensional model, and the second virtual three-dimensional model is registered to the global reference frame.

5. The method according to claim 1, wherein before the first virtual three-dimensional model is matched with the second virtual three-dimensional model, a local reference frame is established at a center of the first virtual three-dimensional model, and the local reference frame is aligned with the global reference frame.

6. The method according to claim 1, further comprising: displaying a relative position of the endoscopic virtual model to the third virtual three-dimensional model on the render window based on the endoscopic virtual position and superimposing the endoscopic image with the virtual image to display the superimposed image.

7. The method according to claim 1, further comprising: constructing a surgical instrument virtual model based on geometrical parameters of a surgical instrument mounted with a second tracker, tracking the second tracker by the position tracking device in order to provide a surgical instrument virtual position, and displaying a relative position of the surgical instrument virtual model to the third virtual three-dimensional model on the render window based on the surgical instrument virtual position.

8. The method according to claim 1, further comprising: photographing an endoscopic calibration tool having a plurality of marked points by using the endoscope to image the plurality of marked points, identifying the plurality of marked points by a computer algorithm to calculate an intrinsic parameter of the endoscope, and adjusting parameters of a virtual camera on the render window by using the intrinsic parameter.

9. The method according to claim 8, wherein the endoscopic calibration tool is a hemisphere tool, and the plurality of marked points is marked on a curved surface of the hemisphere tool.
